(19) United States
(12) Patent Application Publication
(10) Pub. No.: US 2011/0071294 A1
(43) Pub. Date: Mar. 24, 2011

(54) Homogeneous Dimerization Catalysts Based on Vanadium

(75) Inventors: Julian R.V. Lang, Bayreuth (DE); Helmut G. Alt, Bayreuth (DE); Roland Schmidt, Bartlesville, OK (US)

(73) Assignee: CONOCOPHILLIPS COMPANY - IP SERVICES GROUP, Houston, TX (US)

(21) Appl. No.: 12/831,751

(22) Filed: Jul. 7, 2010

Related U.S. Application Data
(60) Provisional application No. 61/224,023, filed on Jul. 8, 2009.

(51) Int. Cl. C07C 2/02; C07F 19/00

(52) U.S. Cl. 546/12; 585/506

(57) ABSTRACT

A series of new bis(imino)pyridine vanadium(III) complexes was synthesized according to formula:

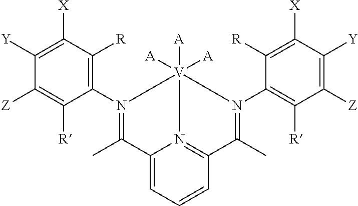

They were tested for the homogeneous catalytic dimerization of propylene after activation with MAO and showed excellent selectivity for dimerization. The catalysts can be used with or without PPh₃ as an additive to produce ≧80% dimerized alkenes.

### CROSS REFERENCE TO RELATED APPLICATIONS

This application claims priority to U.S. Provisional Application No. 61/224,023, filed Jul. 8, 2009.

### FEDERALLY SPONSORED RESEARCH STATEMENT

Not applicable.

### REFERENCE TO MICROFICHE APPENDIX

Not applicable.

### FIELD OF THE INVENTION

The invention relates generally to novel catalysts for the selective dimerization of alkenes.

### BACKGROUND OF THE INVENTION

Unsaturated short chained hydrocarbons are low priced educts for polymerization, oligomerization and metathesis application, produced by unselective thermal cracking processes [1]. Propylene in particular plays an important role for the formation of gasoline with a high octane number. These developments use the selective catalytic dimerization and oligomerization of propylene. On this route branched hexenes can be obtained and used as gasoline blending compounds. The Research Octane Number (RON) rises with the number of branching [2-6], from RON=96-99 for methylpentenes to 101 for dimethylbutene [2, 7-8]. Linear hexenes are in the range from 73-94 and play no role as additives for gasoline improvement. With the ban of lead-alkyl compounds and methyl-tert-butyl ether from gasoline, branched hydrocarbons represent a very important class of compounds for gasoline reformulation [9].

The invention of highly active iron- and cobalt based olefin polymerization and oligomerization catalysts in the late 1990s has led to much interest in the chemistry of transition metal complexes bearing tridentate bis(imino)pyridine ligands [10-18]. These types of complexes were applied by Gibson and Brookhart in 1998 and great progress has been achieved since then. It is well established that bis(imino)pyridine iron(II) complexes (and more recently Fe(III) complexes) show high activities and selectivites for the oligo- and polymerization of ethylene after activation with methyl aluminoxane (MAO). Several complexes with various metal centers and different ligand structures were published and many studies have reported the effects of ligand substitution patterns on activity and selectivity [19]. Bis(imino)pyridine vanadium(III) complexes were found to be selective for the oligomerization of ethylene to give linear olefins [13, 20-22]. These facts underline the importance of such catalysts.

Here we report the application of bis(imino)pyridine vanadium(III) complexes combined with MAO as co-catalyst in the selective dimerization of propylene. The influence of phosphorous containing additives is another aspect in this invention.

### SUMMARY OF THE INVENTION

The invention generally relates to new bis(imino)pyridine vanadium(III) complexes of the general formula:

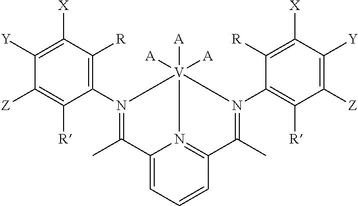

as well as method of making and methods of using said catalysts.

The catalysts are particularly useful for the homogeneous catalytic dimerization of alkenes, particularly with the co-catalyst methyl aluminoxane (MAO). The catalysts can be used with or without triphenylphosphine (aka triphenylphosphane or PPh₃) as an additive to produce ≧80% dimerized alkenes.

In preferred embodiments, R is H or alkyl, X is H, halide or alkyl, Y is H, alkyl, or substituted alkyl or aryl, halide, or oxide, Z is H, alkyl or halide, and R′ is H, alkyl, halide or oxide, A is halide. In other preferred embodiments, R is H, methyl, ethyl, iso-propyl, tert-butyl, propyl, benzyl, or substituted alkyl or aryl, X is F, Cl, Br, H, or methyl Y is methyl, Cl, I, NO₂, butyl, Br, Cl, F or H, Z is H, Br, methyl, and R′ is H, methyl, iso-propyl, or substituted alkyl or aryl, or Cl. In highly preferred embodiments, the catalysts are catalysts 2-4, 8, 12, 14-18, 20, 23, 26 and 27 of Table 1, and particularly preferred are catalysts 2, 3, 14-7 of Table 1.

A method of dimerizing an alkene is also provided, comprising reacting one or more of the catalysts above with MAO and an alkene to produce at least 80% dimerized alkene. In preferred embodiments, at least 85%, 90%, or 95% dimers are formed. In further preferred embodiments, comprise adding triphenylphosphine or other aryl or alkyl substituted triphosphines to the polymerization reaction.

### BRIEF DESCRIPTION OF THE DRAWINGS

FIG. 1 is graph of Scheme 4 showing catalysts 2-4,8,12, 14-18, 20, 23, 26 and 27 with the highest selectivity towards dimerization products of propylene.

FIG. 2 is graph of Scheme 5 showing the product distribution of the reaction of the complexes 17 and 26 and propylene with a various ratio of the additive PPh₃.

### DESCRIPTION OF EMBODIMENTS OF THE INVENTION

The bis(imino)pyridine ligand precursors were synthesized via a condensation reaction (Scheme 1) of 2,6 diacetylpyridine with the respective aniline according to the literature [23].

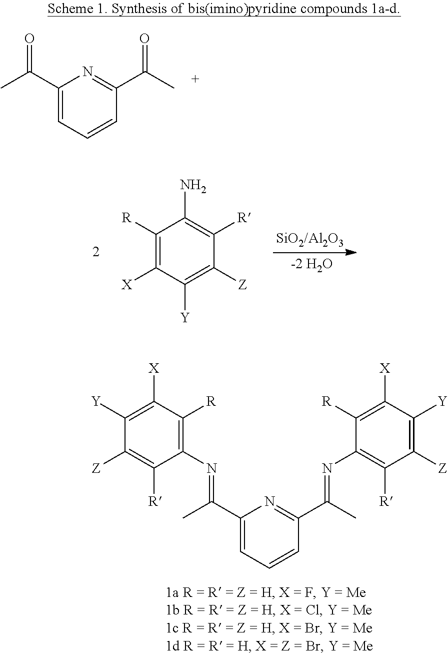

The yields of the compounds 1a-d were generally high (up to 94%).

The complexes were then synthesized via an addition reaction (Scheme 2) of the vanadium(III) trichloride THF adduct and the respective bis(imino)pyridine compound in diethyl ether. The resulting complexes were obtained in good yields (65-87%), in the case of A=Cl.

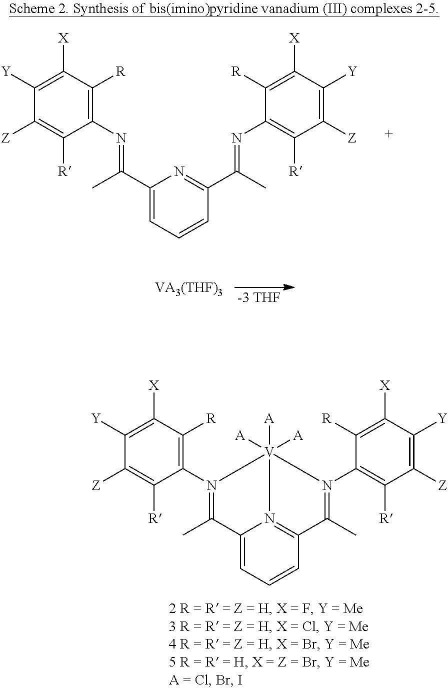

The listed complexes 2-28 were all tested for their catalytic activity in dimerization reactions (Table 1).

TABLE 1
Synthesized complexes 2-28, A = Cl.

| V(III) complex | | | | | |
|---|---|---|---|---|---|
| no. | R | X | Y | Z | R′ |
| 2 | H | F | methyl | H | H |
| 3 | H | Cl | methyl | H | H |
| 4 | H | Br | methyl | H | H |
| 5 | H | Br | methyl | Br | H |
| 6 | H | H | Cl | H | H |
| 7 | H | H | I | H | H |
| 8 | H | H | NO₂ | H | H |
| 9 | methyl | H | I | H | H |
| 10 | methyl | H | methyl | H | methyl |
| 11 | methyl | H | H | H | H |
| 12 | ethyl | H | H | H | H |
| 13 | iso-propyl | H | H | H | H |
| 14 | tert-butyl | H | H | H | H |
| 15 | propyl | H | H | H | H |
| 16 | benzyl | H | H | H | H |
| 17 | iso-propyl | H | H | H | methyl |
| 18 | iso-propyl | H | H | H | iso-propyl |
| 19 | methyl | H | methyl | H | H |
| 20 | H | H | butyl | H | H |
| 21 | methyl | methyl | H | H | H |
| 22 | methyl | H | H | H | Cl |
| 23 | methyl | H | H | methyl | H |
| 24 | H | H | Br | H | H |
| 25 | methyl | H | Cl | H | H |
| 26 | methyl | H | H | H | methyl |
| 27 | H | H | F | H | H |
| 28 | methyl | Cl | H | H | H |

Various bis(imino)pyridine vanadium(III) compounds were tested for the dimerization of propylene after activation with MAO (V:Al=1:500) to give hexene isomers. The catalytic activities and selectivities of the corresponding catalysts are important aspects of a desired catalyst.

The activity was determined by the weight increase of the reaction vessel after removing the propylene. While high activities for the oligo- and polymerization of ethylene were achieved with this type of catalyst [21, 24], the results with propylene varied in the range of 95-215 kg/mol h. For our application, it is more important to have a look at the selectivities and product distributions.

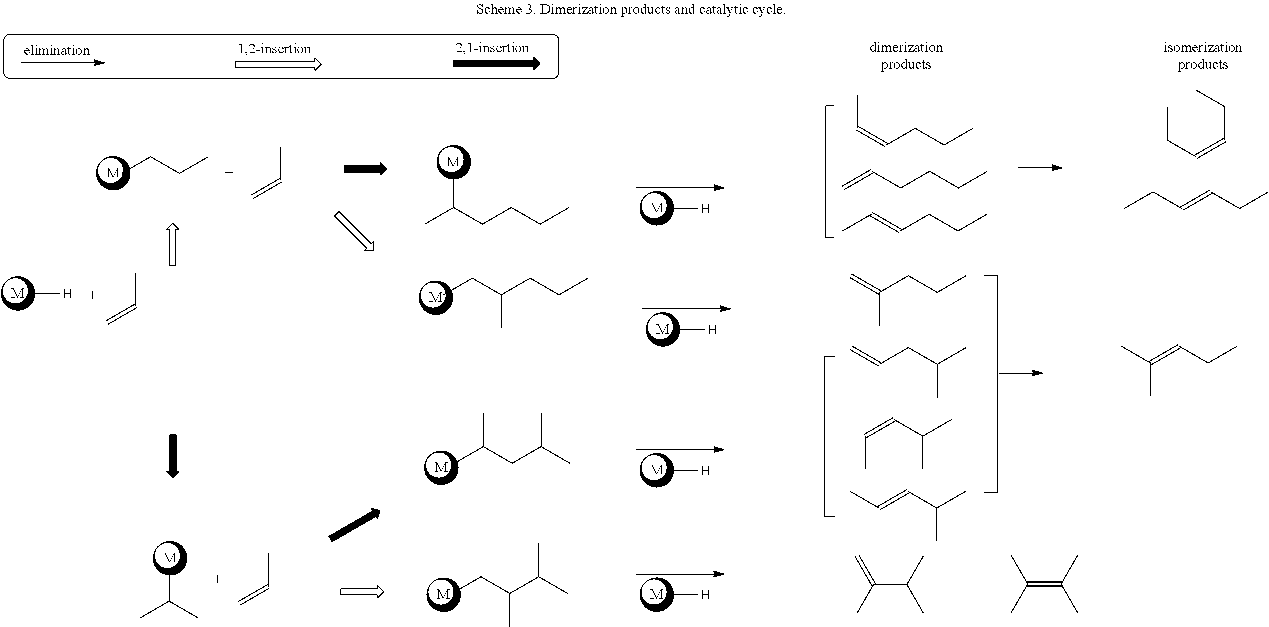

The dimerization of propylene can lead to 12 hexene isomers via coordination, double insertion and elimination reactions (Scheme 3).

It is obvious that complexes 14-17 with bulky ligands like alkyl/aryl substitution on positions 2 or 6 (ortho position) of the imine fragment, achieve high selectivities up to 95% (16). Bulky substituents on both sides have a negative effect. The selectivity falls from 90 to 81% with the replacement of methyl (17) to iso-propyl (18). Moreover, steric hindrance in ortho position has an influence on the product distribution. While complexes 11-13 produce 4-methylpentene (4-MP) as main product, bulky substituents shift it to 2-methylpentene (2-MP). These bulky groups favor 1,2-insertion as an initial step.

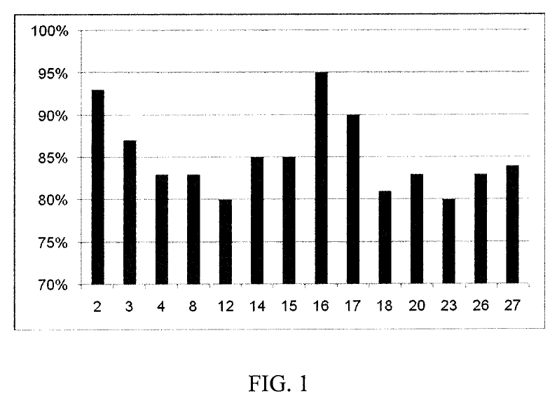

A substitution with halides on the para position has a great influence on the formation of hexenes. Compared to complex 11 (main product 4-MP-1 with a selectivity of 62%), a halide substitution gives 4-MP-1 with selectivities between 74% (25) and 82% (9). See FIG. 1.

The selectivity of the formation of hexene isomers decreases in the following manner F (93%) (2) >Cl (87%) (3) >Br (83%) (4) on the meta position. The β-hydrogen elimination is favored by electron withdrawing groups compared to the heavier homologue halides. The distribution of the dimeric products is nearly the same for all three halide substituted complexes with 4-MP-1 as main product and selectivities up to 90% are observed. With the high dimer and product selectivity of 2,4-MP-1 is produced with a total amount of 83%.

Electron withdrawing or pushing groups on position 4 of the imine fragment have no influence on the dimer selectivity (6-8, 20, 24 and 27). The difference is obvious in product distribution. Complex 20 with a withdrawing group produces 2-MP-1 with 47%. On the other side, electron pushing groups generate 4-MP-1 with an amount of up to 75%.

The kind of substitution at the meta position of the bis(imino)pyridine complex has no influence on the selectivity of the dimers, but it effects the distribution of the dimers immensely. Complexes 6-9, 24, 25 and 27 with a −J-effect at the meta position of the phenyl group give a maximum selectivity of 2-MP-1 of 13%. A ligand with a +I-effect at the same position give complex 20 which shows a selectivity for 2-MP-1 of 47%. The formation of 4-MP-1 shows its highest selectivity (90%) (2) in contrast to the formation of 2-MP-1 by the reaction of complexes with a −I-effect at the ligand precursor like Cl, Br or J.

These two products are generated by different first insertion steps, and are caused by the electronic influence of both substituents. Complex 5 is the only complex that produces 2,3-DMB-1 in satisfying yields (25%) with medium selectivity towards dimerization products.

TABLE 2
Selectivity of dimerization products and product distribution
within hexene isomers for the vanadium(III) complexes 2-28

| V(III) | Selectivity | Products within the dimers (%) | | | | | | | |
|---|---|---|---|---|---|---|---|---|---|
| complex no. | to dimers (%) | 4-MP-1 | 2,3-DMB-1 | c-4-MP-2 | t-4-MP-2 | 2-MP-1 | t-2-hex | 2-MP-2 | c-2-hex |
| 2 | 93 | 90 | 1 | 4 | — | 5 | — | — | — |
| 3 | 87 | 85 | 2 | 6 | 3 | 4 | — | — | — |
| 4 | 83 | 89 | 1 | 7 | 1 | 2 | — | — | — |
| 5 | 60 | 24 | 25 | 45 | 0 | 6 | — | — | — |
| 6 | 70 | 68 | 5 | 14 | 3 | 9 | — | 1 | — |
| 7 | 72 | 71 | 2 | 13 | 2 | 8 | — | 4 | — |
| 8 | 83 | 73 | 5 | 3 | 3 | 13 | — | 3 | — |
| 9 | 75 | 82 | — | 9 | 3 | 6 | — | — | — |
| 10 | 55 | 73 | — | 7 | — | 20 | — | — | — |
| 11 | 55 | 62 | 2 | 18 | 4 | 14 | — | — | — |
| 12 | 80 | 68 | 2 | 14 | 5 | 10 | 1 | — | — |
| 13 | 60 | 55 | — | 13 | 3 | 26 | 3 | — | 1 |
| 14 | 85 | 5 | — | 8 | 11 | 75 | 1 | — | — |
| 15 | 85 | 36 | — | 10 | 6 | 46 | 1 | 1 | — |
| 16 | 95 | 7 | — | 7 | 6 | 80 | — | — | — |
| 17 | 90 | 3 | — | 4 | 5 | 88 | — | — | — |
| 18 | 81 | 11 | — | 5 | 7 | 77 | — | — | — |
| 19 | 75 | 8 | — | 7 | 9 | 76 | — | — | — |
| 20 | 83 | 19 | 5 | 15 | 7 | 47 | 1 | 6 | 1 |
| 21 | 76 | 34 | 1 | 10 | 8 | 45 | 1 | 1 | 1 |
| 22 | 70 | 32 | 2 | 10 | 4 | 52 | — | — | — |
| 23 | 80 | 25 | — | 7 | 5 | 63 | — | — | — |
| 24 | 77 | 70 | — | 17 | 2 | 6 | — | 5 | — |
| 25 | 40 | 74 | 1 | 13 | 4 | 8 | — | — | — |
| 26 | 83 | 41 | — | 5 | 3 | 51 | — | — | — |
| 27 | 84 | 75 | 3 | 9 | 3 | 6 | — | 4 | — |
| 28 | 77 | 72 | 2 | 14 | 6 | 6 | — | — | — |

In the late 1960's, Wilke recognized the influence of additives in catalytic reactions [25]. Phosphanes are widely used additives and a positive influence on selectivity and activity was observed during dimerization of propylene [26]. We tested triphenylphosphine (aka triphenylphosphane), which is a common organophosphorus compound with the formula P(C₆H₅)₃ (abbreviated PPh₃) for use with the invention.

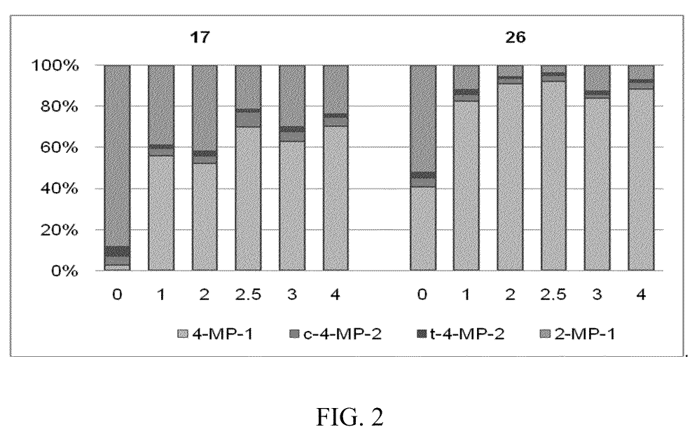

The relevant complexes were dissolved in toluene, PPh₃ was added in a ratio of metal:additive=1:1, (2, 2.5, 3 and 4) stirred for 30 min and activated with MAO. See FIG. 2.

The addition of the additive had a positive influence on the dimer selectivity (90%) with the use of 2 eq. PPh₃ for 17. The selectivity could be improved up to 95%. For all other amounts no improvement could be detected. In contrast, the use of additive had great influence on the product distribution. With the addition of 2.5 equiv. a maximum of 70% for the formation of 4-MP-1 (17) could be achieved. The absence of PPh₃ effects the formation of 2-MP-1 with a selectivity of 88%. Insertion mechanisms are influenced by the use of phosphine containing additives, which results in an 1,2-insertion instead of 2,1-insertion. The results of the corresponding reactions of complex 26 confirm the additive dependency as discussed before. A selectivity of 90% was detected for 4-MP-1 by the addition of 2-2.5 mole PPh₃ in contrast to 51% without an additive.

Novel complexes of the type bis(imino)pyridine vanadium(III) (2-5) were synthesized. Because of the simple synthetic route, numerous substitution patterns can be performed. Bulky substituents on the ortho position have positive influence on the selectivity of the dimer products. Complex 16 with a benzyl substituent at the ortho position gave a selectivity of 95% for dimers. Substituents at the 2 and 6 positions of the phenyl group accrue the 1,2-propylene insertion. Different halide groups as substituents on the para position have no influence on the product distribution and selectivity. Effects can be obtained when electron withdrawing and donating groups are introduced. The first ones generate 4-MP-1 as main product. Electron pushing substituents give 2-MP-1. The octane numbers of the main products are between 94% and 99%. It is obvious, that the structure of the precatalyst, in particular the substitution pattern of the organic compound, has a great influence on the product distribution, but not on the selectivity. No dependence for dimer selectivity is obvious from the insertion pathway. In less cases the expected multiple branched hexenes could be obtained. Complex 5 produced 2,3-DMB-1 in yields of 25% within the dimerization products. The use of additives had a positive influence on the product distribution and was very selective for complex 26. Complex 26 and 2 equiv. of the additive PPh₃ produced 90% of 4-MP-1 within the dimers. In the case of complex 17 the use of an additive had an enormous effect on the initial insertion step. It changed from 90% of 1,2-insertion up to 78% for 2,1-insertion with the use of 2.5 equiv. of PPh₃.

### Example 1

### Experimental

Air- and moisture sensitive reactions were carried out under an atmosphere of purified argon using conventional Schlenk or glove box techniques. The dimerization reactions were performed with pressure Schlenk tubes.

The products of the dimerization experiments were characterized by a gas chromatograph (AGILENT™ 6890) and GC/MS (FOCUS DSQ™ THERMO SCIENTIFICT™). Mass spectra were recorded on a VARIAN™ MAT CH7 instrument (direct inlet system, electron impact ionization 70 eV). Elemental analyses were performed with a VARIOEL™ III CHN instrument. Acetanilide was used as standard. NMR spectra were taken on a VARIAN INOVA™ 400 instrument. The samples were prepared under argon atmosphere and measured at room temperature. Chemical shifts (6, ppm) were recorded relative to the residual solvent peak at δ=7.24 ppm for chloroform-d. The multiplicities were assigned as follows: s, singlet; m, multiplet; t, triplet. ¹³C {¹H} NMR spectra were fully proton decoupled and the chemical shifts (δ, ppm) are relative to the solvent peak (77.0 ppm).

All solvents were purchased as technical grade and purified by distillation over Na/K alloy under an argon atmosphere. All other chemicals were purchased commercially from ALDRICH™ or ACROS™ or were synthesized according to literature procedures. The methyl aluminoxane solution (MAO, 30 wt. % in toluene) was obtained from ALBEMARLE™, USA.

10 g mole sieves (4A) and 0.5 g of catalytically active SiO₂/Al₂O₃ pellets were added to a solution of 0.49 g (3.0 mmol) diacetylpyridine in toluene. After addition of 7.0 mmol of the respective aniline, the solution was heated at 45° C. for 24 hours. After filtration over Na₂SO₄ and evaporation to dryness, the products were precipitated as yellow solids from methanol overnight at −20° C. (73-94%).

Spectroscopic data: 1a: 1H NMR (400 MHz, CDCl₃): 8.30 (d, 2H, Py-Hm), 7.85 (t, 1H, Py-Hp), 7.15 (t, 2H, Ph-H), 6.53 (m, 4H, Ph-H), 2.39 (s, 6H, N═CMe), 2.26 (s, 6H, Ph-CH3). 13C {1H} (100.5 MHz, CDCl₃): 167.9 (Cq), 163.1 (Cq), 159.9 (Cq), 155.3 (Cq), 150.4 (Cq), 136.9 (CH), 131.6 (CH), 122.4 (CH), 114.8 (CH), 106.6 (CH), 16.2 (CH3), 14.1 (CH3). MS data: 377 (M^{•+}) (88), 362 (12), 150 (100).

Spectroscopic data: 1b: 1H NMR (400 MHz, CDCl₃): 8.30 (d, 2H, Py-Hm), 7.8t (t, 1H, Py-Hp), 7.21 (d, 2H, Ph-H), 6.87 (s, 2H, Ph-H), 6.64 (d, 2H, Ph-H), 2.40 (s, 6H, N═CMe), 2.36 (s, 6H, Ph-CH3). 13C {1H} (100.5 MHz, CDCl3): 168.0 (Cq), 155.3 (Cq), 150.1 (Cq), 134.5 (Cq), 130.9 (Cq), 136.8 (CH), 131.2 (CH), 122.4 (CH), 119.8 (CH), 117.8 (CH), 19.4 (CH3); 16.3 (CH3). MS data: 409 (M^{•+}) (52), 166 (100).

Spectroscopic data: 1c: 1H NMR (400 MHz, CDCl₃): 8.30 (d, 2H, Py-Hm), 7.85 (t, 1H, Py-Hp), 7.21 (d, 2H, Ph-H), 7.06 (s, 2H, Ph-H), 6.70 (d, 2H, Ph-H), 2.40 (s, 6H, N═CMe), 2.39 (s, 6H, Ph-CH3). 13C {1H} (100.5 MHz, CDCl₃): 168.1 (Cq), 155.2 (Cq), 150.1 (Cq), 132.7 (Cq), 124.9 (Cq), 136.8 (CH), 131.0 (CH), 123.0 (CH), 122.4 (CH), 118.4 (CH), 22.2 (CH3), 16.3 (CH3). MS data: 499 (M^{•+}) (52), 484 M—Me (8), 210 CH3C═NAr (100).

Spectroscopic data: 1d: 1H NMR (400 MHz, CDCl₃): 8.48 (d, 2H, Py-Hm), 8.07 (t, 1H, Py-Hp), 7.24-7.44 (m, 4H, Ph-H), 2.76 (s, 6H, Ph-CH3), 2.62 (s, 6H, N═CMe). 13C {1H} (100.5 MHz, CDCl₃): 169 (Cq), 155 (Cq), 151 (Cq), 132.0 (Cq), 125.2 (Cq), 137.0 (CH), 129 (CH), 122.7 (CH), 23.0 (CH3), 16.5 (CH3). MS data: 657 (M^{•+}) (52), 577 M—Br (17), 290 M—CH3C═NAr (100).

An amount of 0.22 mmol of the respective bis(imino)pyridine compound was dissolved in 20 ml diethylether and stirred. A stoichiometric amount of vanadium trichloride-tetrahydrofuran adduct was added at room temperature. Stirring was continued overnight. Pentane was added to precipitate the product, which was subsequently collected by filtration, washed with pentane and dried in vacuo. The resulting solids were obtained with an overall yield of 65-87%.

Spectroscopic data: 2: MS data: 533 (M^{•+}) (8), 497 M-Cl (100), 377 (30), 150 (62), 36 (100). C₂₃H₂₁Cl₃F₂N₃V (533.02): calcd. C, 51.66; H, 3.96; N, 7.86. Found C 49.87, H 4.34, N 7.02%.

3: MS data: 565 (M^{•+}) (13), 531 M—Cl (100), 406 (18), 396 (10). C₂₃H₂₁Cl₅N₃V (564.96): calcd. C, 48.67; H, 3.73; N, 7.40. Found C, 48.97; H, 3.55; N, 7.13%.

4: MS data: 653 (M^{•+}) (7), 619 (37), 541 (10), 187 (63), 36 (100). C23H21Cl3Br2N3V (652.86): calcd. C, 42.08; H, 3.22; N, 6.40. Found C, 42.61; H, 3.33; N, 6.42%.

5: MS data: 808 (M^{•+}) (4), 772 (100). C₂₃H₁₉Cl₃Br₄N₃V (808.68): calcd. C 33.92, H 2.35, N 5.16. Found C, 33.45; H, 2.30; N, 4.89%.

The respective complex was dissolved in toluene and activated with MAO solution (V:Al=1:500) and transferred into a 400 ml pressure Schlenk tube. The pressure Schlenk tube was filled with 50 ml liquid propylene and closed, warmed to room temperature with an external water bath and stirred. After the reaction time of 1 hour, the Schlenk tube was opened and the solution was analyzed by GC.

The following references are each incorporated by reference in their entirety.
[1] T. Sakakura, T. Sodeyama, M. Tanaka, New J. Chem. 13 (1989) 737.
[2] J. H. Gary, G. H. Handwerk, Petroleum Refining: Technology and Economics, Dekker, New York (1994).
[3]Reference Data for Hydrocarbons and Organosulfur Chemicals, Phillips Petroleum Company (1998).
[4] W. Keim, New J. Chem. 11 (1987) 531.
[5] F. Benvenuti, Appl. Catal. 204 (2000) 7.
[6] S. Wu, S. Lu, J. Mol. Catal. A: Chem. 197 (2003) 51.
[7] C. Carlini, M. Marchionna, A. M Raspolli Galletti, G. Sbrana, J. Mol. Catal. A: Chem. 169 (2001) 19.
[8] Y. Chauvin, H. O. Bourbigou, Chemtech (1995) 26.
[9] M. Marchionna, M. D. Girolamo, R. Patrini, Catalysis Today 65 (2001) 397.
[10] B. L. Small, M. Brookhart, A. M. A. Bennet, J. Am. Chem. Soc. 120 (1998) 4049.
[11] B. L. Small, M. Brookhart, Polym. Preprints 39 (1998) 213.
[12] G. J. P. Britovsek, V. Gibson, B. S. Kimberley, P. J. Maddox, S. J. McTavish, G. A. Solan, A. J. P. White, D. J. Williams, Chem. Commun. (1998) 849.
[13] R. Schmidt, Dissertation, Universitat Bayreuth (1999).
[14] D. Reardon, F. Conan, S. Gambarotta, G. Yap, G. Wang, J. Am. Chem. Soc. 121 (1999) 9318.
[15] R. Schmidt, M. B. Welch, R. D. Knudsen, S. Gottfried, H. G. Alt, J. Mol. Cat. 222 (2004) 9.
[16] R. Schmidt, M. B. Welch, R. D. Knudsen, S. Gottfried, H. G. Alt, J. Mol. Cat. 222 (2004) 17.
[17] B. A. Dorer, WO 047586A1 (2000).
[18] D. D. Devote, S. S. Feng, K. A. Frazier, J. T. Patton, WO 069923A1 (2000).
[19] V. C. Gibson, S. K. Spitzmesser, Chem. Rev. 103 (2003) 283.
[20] J. Romero, F. Carrillo-Hermosilla, A. Antinolo, A. Otero, J. Mol. Cat. 304 (2009) 180.
[21] S. Gottfried, Dissertation, Universitat Bayreuth (2002).
[22] M. J. Hanton, K. Tenza, Organomet. 27 (2008) 5712.
[23] C. Qian, F. Gao, Y. Chen, L. Gao, Synlett 10 (2003).
[24] M. Seitz, Dissertation, Universitat Bayreuth (2004).
[25] G. Wilke, B. Bogdanovic, P. Hardt, O. Heimbach, W. Kroner, W. Oberkirch, K. Tanaka, E. Steinrucke, D. Walter, H. Aimmermann, Angew. Chem. Int. Ed. 5 (1966) 151.
[26] K. Schneider, Dissertation thesis, University Bayreuth (2006).

What is claimed is:

1. An alkene dimerization catalyst having the structure: 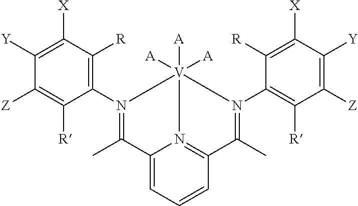
wherein R is H or alkyl;
X is H, halide or alkyl;
Y is H, alkyl, halide, or oxide;
Z is H, alkyl or halide;
R′ is H, alkyl, halide or oxide; and
A is a halide.

2. The catalyst of claim 1, wherein R is H, methyl, ethyl, iso-propyl, tert-butyl, propyl, benzyl, or iso-propyl, or substitute alkyl or aryl;
X is F, Cl, Br, H, or methyl, or substituted alkyl or aryl;
Y is methyl, Cl, I, NO₂, butyl, Br, Cl, F or H, or substituted alkyl or aryl;
Z is H, Br, methyl, or substituted alkyl or aryl;
R′ is H, methyl, iso-propyl, or substituted alkyl or aryl, or Cl; and
A is a halide.

3. The alkene dimerization catalyst of claim 1 wherein R, R′ and Z=H, X=F and Y=methyl.

4. The alkene dimerization catalyst of claim 1 wherein R, R′ and Z=H, X=Cl and Y=methyl.

5. The alkene dimerization catalyst of claim 1 wherein R, R′ and Z=H, X=Br and Y=methyl.

6. The alkene dimerization catalyst of claim 1 wherein R, R′, X and Z=H and Y=NO₂.

7. The alkene dimerization catalyst of claim 1 wherein R=ethyl and X, Y, Z, and R′=H.

8. The alkene dimerization catalyst of claim 1 wherein R=tert-butyl and X, Y, Z, and R′=H.

9. The alkene dimerization catalyst of claim 1 wherein R=propyl and X, Y, Z, and R′=H.

10. The alkene dimerization catalyst of claim 1 wherein R=benzyl and X, Y, Z, and R′=H.

11. The alkene dimerization catalyst of claim 1 wherein R=iso-propyl and X, Y, and Z=H and R′=methyl.

12. The alkene dimerization catalyst of claim 1 wherein R=iso-propyl and X, Y, and Z=H and R′=iso-propyl.

13. The alkene dimerization catalyst of claim 1 wherein R=iso-propyl and X, Y, and Z=H and R′=methyl.

14. A method of dimerizing an alkene comprising reacting the catalyst of claim 3 with methyl aluminoxane (MAO) and an alkene to produce at least 80% dimerized alkene.

15. A method of dimerizing an alkene comprising reacting the catalyst of claim 4 with methyl aluminoxane (MAO) and an alkene to produce at least 80% dimerized alkene.

16. A method of dimerizing an alkene comprising reacting the catalyst of claim 5 with methyl aluminoxane (MAO) and an alkene to produce at least 80% dimerized alkene.

17. A method of dimerizing an alkene comprising reacting the catalyst of claim 6 with methyl aluminoxane (MAO) and an alkene to produce at least 80% dimerized alkene.

18. A method of dimerizing an alkene comprising reacting the catalyst of claim 7 with methyl aluminoxane (MAO) and an alkene to produce at least 80% dimerized alkene.

19. A method of dimerizing an alkene comprising reacting the catalyst of claim 8 with methyl aluminoxane (MAO) and an alkene to produce at least 80% dimerized alkene.

20. A method of dimerizing an alkene comprising reacting the catalyst of claim 9 with methyl aluminoxane (MAO) and an alkene to produce at least 80% dimerized alkene.

21. A method of dimerizing an alkene comprising reacting the catalyst of claim 10 with methyl aluminoxane (MAO) and an alkene to produce at least 80% dimerized alkene.

22. A method of dimerizing an alkene comprising reacting the catalyst of claim 11 with methyl aluminoxane (MAO) and an alkene to produce at least 80% dimerized alkene.

23. A method of dimerizing an alkene comprising reacting the catalyst of claim 12 with methyl aluminoxane (MAO) and an alkene to produce at least 80% dimerized alkene.

24. A method of dimerizing an alkene comprising reacting the catalyst of claim 13 with methyl aluminoxane (MAO) and an alkene to produce at least 80% dimerized alkene.

25. The method of claim 14 wherein at least 90% dimerized alkene is produced.

26. The method of claim 15 wherein at least 90% dimerized alkene is produced.

27. The method of claim 16 wherein at least 90% dimerized alkene is produced.

28. The method of claim 17 wherein at least 90% dimerized alkene is produced.

29. The method of claim 18 wherein at least 90% dimerized alkene is produced.

30. The method of claim 19 wherein at least 90% dimerized alkene is produced.

31. The method of claim 20 wherein at least 90% dimerized alkene is produced.

32. The method of claim 21 wherein at least 90% dimerized alkene is produced.

33. The method of claim 22 wherein at least 90% dimerized alkene is produced.

34. The method of claim 23 wherein at least 90% dimerized alkene is produced.

35. The method of claim 24 wherein at least 90% dimerized alkene is produced.

36. The alkene dimerization catalyst of claim 1 wherein R, X, Z, and R′=H and Y=butyl.

37. The alkene dimerization catalyst of claim 1 wherein R=methyl; X, Y, and R′=H and Z=methyl.

38. The alkene dimerization catalyst of claim 1 wherein R and R′=methyl; and X, Z, and Y=H.

39. The alkene dimerization catalyst of claim 1 wherein R, X, Z and R′=H and Y=F.

40. A method of dimerizing an alkene comprising reacting the catalyst of claim 36 with methyl aluminoxane (MAO) and an alkene to produce at least 80% dimerized alkene.

41. The method of claim 40 wherein at least 90% dimerized alkene is produced.

42. The method of claim 14, further comprising adding triphenylphosphine or substituted triphenylphosphine to said reaction.

43. A method of making the catalyst of claim 1 comprising performing the following reactions: [chemical structure]
